(19) United States
(12) Patent Application Publication
(10) Pub. No.: US 2006/0275238 A1
(43) Pub. Date: Dec. 7, 2006

(54) Topical cosmetic formulations for regulating and improving the moisture content of the skin

(75) Inventors: Heike Blasko-Begoihn, Essen (DE); Mike Farwick, Essen (DE); Peter Lersch, Dinslaken (DE); Ursula Maczkiewitz, Essen (DE); Maria Mecking, Bottrop (DE); Ralph Scheuermann, Essen (DE); Christian Weitemeyer, Essen (DE)

Correspondence Address: Leopold Presser, Scully, Scott, Murphy & Presser, 400 Garden City Plaza, Garden City, NY, 11530, US

(73) Assignee: Goldschmidt GmbH, Essen (DE)

(21) Appl. No.: 11/441,659

(22) Filed: May 26, 2006

(30) Foreign Application Priority Data
Jun. 7, 2005 (DE) 10 2005 026 069.1

(51) Int. Cl. A61K 8/73; A61K 8/64

(52) U.S. Cl. 424/70.13; 424/70.14

(57) ABSTRACT

Cosmetic and/or dermatological formulations for the topical treatment of the skin for regulating and improving the moisture content of the skin, comprising as active ingredient combination a) at least one anionic polyamino acid matrix, b) at least one polysaccharide, and optionally c) one or more osmoprotectants for topical application are provided.

### FIELD OF THE INVENTION

The present invention relates to novel cosmetic formulations for the local topical treatment of parts of the body that have a content of substances which increase skin moisture, which are intended to maintain and to treat the epidermal horny layer, in particular, whilst simultaneously ensuring hydration, a lasting improvement in the water balance and a reduction in the roughness of the skin.

### BACKGROUND OF THE INVENTION

The horny skin, which constitutes the outermost layer of the skin, is an important barrier layer and is of particular importance for protection against environmental influences. Besides lipids, the skin also requires an optimum of water to retain its smoothness, elasticity and suppleness. These findings were confirmed in the basic works, inter alia, by Jacobi, and Schuleit and Szakall. A person releases several deciliters, up to several liters of water daily, into the environment by the skin. The water located in the skin originates from various sources and, according to relatively recent findings, is present either as vapor or else in liquid form, and also adsorbed to proteins. It is not known how much water the epidermis contains. Since some layers in the horny layer contain up to 70% proteins and lipids, water of up to 30% may be assumed. What can be assumed with certainty is that water can migrate through different layers in the skin.

There are various models for the diffusion of water through the skin layers, although none has as yet been proven conclusive.

Analogous to hydrophobic substances which can penetrate through lipid pores in the horny layer (stratum corneum, SC), the water is said to be transported by specific “aqueous pores”. These pores are said to have a diameter from 15 to 25 Å.

Another approach postulates that water-filled channels pass through the stratum corneum. Using diffraction experiments with X-rays, it has been shown that there are holes in a lipid double-layer system which are large enough to collect water condensed therein.

For regulating moisture in the skin, besides an intact permeability barrier, the presence of water-binding substances which are formed in the epidermal horny layers is thus also undoubtedly required. The natural moisturizing factors (NMFs) present in the epidermis bind moisture in the skin. The NMFs constitute a mixture of different compounds and consist of 40% amino acids, 12% pyrrolidonecarboxylic acid, 7% urea, and 41% inorganic and organic salts (primarily lactates).

An increased transepidermal water loss (TEWL) causes the skin to dry out. It is assumed that the loss of the natural moisturizing factors correlates with a reduction in the water content and a reduced softness of the keratin layer.

Drastic environmental conditions, such as low temperatures or too little moisture in the winter, contribute to a considerable degree to the skin becoming rough and dry. The moisturizing factors present in the epidermis are likewise readily removed by frequent washing or bathing, especially in cases of the simultaneous action of washing or cleaning compositions. Thus, more water can escape from deeper skin layers and the transepidermal water loss increases. Sensorily, this manifests itself through symptoms such as, for example, a more rough, flaky, lackluster and dull-looking skin surface. Loss of flexibility and impairment of the barrier function of the skin, which depends on the water-binding capacity of the horny layer, are the result. Consequently, the water content of the horny layer is further reduced.

Moisturizing and moisture regulation of the skin and also careful care for preventing constantly dry skin are not only an esthetic requirement, but also a tried and tested means of effectively preventing chronic skin diseases.

A large number of in vivo methods with which the moisture content of the skin can be determined are known. Here, particularly conductivity and the dielectric properties (capacity) of the horny layer are used for determining corneometric skin moisture. Available instruments with the help of which water content of the stratum corneum can be determined include the corneometer models CM 820 and CM 825 (Courage+Khazaka), and the dermal phase meter Skicon 200 (Nova). These noninvasive and simple methods allow the change in horny layer hydration to be quantitatively measured.

The viscoelasticity of the skin can be determined via the dermal torque meter (DiaStron) or else via the cutometer (Courage+Khazaka).

There are a number of cosmetic preparations with a hydroregulative effect for counteracting a dry skin state and restoring the water balance in the skim These prior art preparations are, in the form of emulsions, ideal preparations for supplying the skin with fat and moisture. The prior art preparations generally comprise a number of active ingredients which, upon application, develop a protective function, thereby improving the condition of the skin surface and changing the functional state of the skin by, for example, having a regulating effect on the skin moisture, and care properties taking effect as a result of penetration under the surface of the skin. There are various mechanisms as to how the water content of the epidermis can be positively influenced by cosmetic ingredients and formulations.

The evaporation of water from the upper skin layers can be suppressed by an occlusive lipid or polymer film. As a result, water is released from the lower skin layers to the upper skin layers, and the formation of perspiration is reduced, as a result of which the skin moisture of the upper layers of the SC increases considerably. However, under such occlusive conditions, the result is typically a build-up of water in the skin and increased endogenous swelling of the horny layer, as a result of which the regeneration ability of the skin is slowed.

From a formulation point of view, it is possible to produce cosmetic products which contain more water than the external skin layers and thus release water to the stratum corneum. Specific lipids are likewise able to reduce the transepidermal water loss and can therefore also be regarded as being a type of moisturizer.

A further customary approach is the addition of moistures as activating ingredients to cosmetic emulsions, which are intended to ensure that the keratin layer is supplied with an adequate amount of moisture over defined time intervals. Moisturizers are also referred to as humectants and are intended, on the one hand, to retain water in the epidermis, and on the other hand, to reduce the TEWL by stabilizing the barrier function in the upper horny layer.

A large number of such substances have been described and are already used. These are generally hygroscopic substances with the ability to bind water to a greater or lesser degree and to completely or partially replace the washed-out natural substances. In principle, the hygroscopic substances include, for examples, polyhydric alcohols, ethoxylated polyols, sugars, and polysaccharides, amino acids and protein hydrolysates.

The most important representatives of these moisturizers include urea, which is an effective moisturizer with good water-binding capacity. In addition, urea can promote the permeation of additional active ingredients into the skin Besides urea, the essential amino acid arginine is also a type of natural moisturizing factor. Also known is an active system of arginine, urea and glycerol, which brings about a hydrating effect in the horny layer and helps to combat and prevent skin dryness.

A further important representative is glycerol (glycerin, 1,2,3-propanetriol). It is assumed that glycerol remains on or close to the surface of the skin. If the ambient atmospheric humidity is very low, water is released through the skin to the outside. The glycerol replaces this water loss by absorbing water from the lower skin layers, i.e., it stimulates the circulation of water through the epidermis. Recent publications explain the moisturizer effect of glycerol with its ability to promote certain enzyme reactions which are required for restoring an intact SC.

A likewise very effective moisturizer is hyaluronic acid and its salts. Hyaluronic acid is a polymeric glycosaminoglucan which is present in associated from with other tissue components and is important for controlling tissue hydration. Hyaluronic acid has an enormous water-binding capacity and holds the water in the stratum corneum, as a result of which the elasticity and softness of the skin is increased. A decreasing content of hyaluronic acid in the skin correlates with skin aging and wrinkle formation.

There has thus hitherto been no lack of attempts to positively influence the moisture in the skin by adding ingredients, and in the past a large number of different substances has already been proposed which, for their part, intervene in different regulation mechanisms of skin moisture regulation. For example, the patent literature describes a series of moisture-binding substances and balanced mixtures which have a regulating effect on skin moisture.

Examples of the use of amino acids as moisturizers are found in U.S. Pat. Nos. 4,772,591, 3,849,576, 5,135,913, 4,859,653 and 4,760,051, and in the European patent specification EP-B 0 070 048. These describe specific vitamins and amino acids either on their own or in a multitude of combinations. Also included therein are certain formulations which describe certain amino acid complexes which are formed from the amino acids such as proline, arginine, pyrrolidonecarboxylic acid or glycine and are intended to serve to penetrate into the skin in order to retain the skin moisture and to promote homeostasis of the epidermis and stimulate cell growth.

WO-A-00/15187 (SKW Trostberg) discloses the use of creatine as a moisturizer in cosmetic preparations. The use of creatine or suitable derivatives thereof is said to overcome symptoms of dry skin such as chapping and flaking with a lasting effect.

U.S. Pat. No. 5,254,331 describes a skin cream which comprises a protein-amino acid-vitamin-nucleotide complex. This complex consists of propylene glycol, serum proteins, niacinamide, water, adenosine phosphate and arginine. These constituents are said to stimulate regeneration of the epidermal cells and improve skin moisture continuously and with a lasting effect.

U.S. Pat. No. 5,215,759 describes cosmetic aqueous formulations which comprise skin-moisturizing mixtures comprising 20 to 40% glycerol, 10 to 20% chitin, 5 to 15% sodium lactate, 1 to 5% sodium chloride, 5 to 15% sodium pyrrolidonecarboxylate, in each case 1 to 5% glycogen, urea and propylene glycol, and also at least 1%, in each case, of an amino acid selected from a group of amino acids which includes glycine, arginine, lysine, histidine and ornithine or placenta protein, as constituents.

Preferably, such a substance should bring about a significant effect even in low use concentrations, should be nontoxic, preferably of natural origin, be very well tolerated by the skin, have high compatibility with other ingredients, have good long-term stability and be able to be incorporated into skin-treatment compositions without problems.

It is particularly desirable if these substances can additionally be prepared easily and cost-effectively and are produced in a form which can be purified easily and thus satisfies the high purity conditions placed on cosmetic and/or dermatological active ingredients. In addition, the substances used should have multifunctional properties, thus besides normalizing the water content of the skin, in addition also have protective, calming and free-radical-scavenging properties.

Despite many years search in the field of skin moisturizers, the majority of users find that, upon closer inspection, the substances currently used as moisturizers do not entirely meet the requirements placed on them.

### SUMMARY OF THE INVENTION

The underlying objective of the invention is to prepare such an active ingredient combination which is not burdened by the disadvantages mentioned above. Another objective of the present invention is to provide an active ingredient combination which has improved skin and mucosa compatibility and is able to increase the water-binding capacity in the epidermis and on the surface of the skin, the effect of which can be adjusted in a targeted manner over a prolonged period and at the same time covers the skin with a protective film of biodegradable substances, the metabolites of which supply the skin with nutrients and, overall, effectively assist the skin in its restructuring process.

The present invention thus provides cosmetic and/or dermatological formulations for the topical treatment of the skin for regulating and improving the moisture content of the skin, comprising as an active ingredient combination
a) at least one anionic polyamino acid matrix,
b) at least one polysaccharide, and optionally
c) one or more osmoprotectants
   for topical application.

The present invention further provides cosmetic and/or dermatological formulations for the topical treatment of the skin which simultaneously form an interpenetrating network for the controlled, delayed release of the incorporated active ingredients.

The present invention further provides cosmetic and/or dermatological formulations for the topical treatment of the skin which exhibit hysteresis behavior with regard to water binding, i.e., resulting in delayed release of the bound water, both on the surface of the skin, and also on the epidermis.

### BRIEF DESCRIPTION OF THE DRAWINGS

FIGS. 1-7 are graphs ploting the weight increase, %, vs. relative atmospheric humidity, %, for various moisturizers mentioned in Example 4.

FIG. 8 is a graph ploting the delta CU vs. time, min., for a blank formulation and the active ingredient combination 1 in accordance with the description presented in Example 5.

FIG. 9 is a graph showing the increase in the relative CU by the active ingredient combination 1 compared to a blank sample in accordance with the description presented in Example 6.

FIG. 10 is a graph showing the roughness of the skin and appearance of the skin using the active ingredient combination 1 as also described in Example 6.

FIG. 11 is a graph showing the improvement in the skin treated with the active ingredient combination 1 compared with skin treated with a blank sample also in accordance with the description of Example 6.

### DETAILED DESCRIPTION OF THE INVENTION

Surprisingly, it has been found that a combination of an anionic polyamino acid matrix with a polysaccharide matrix forms an interpenetrating polymer network. The choice of components for the interpenetrating polymer network ensures that there is high biocompatibility and the resulting hydrophilic moisturizer for cosmetic preparations is also compatible with many other raw materials and auxiliaries. In addition, the polymer network is surprisingly capable of incorporating other auxiliaries and, following application to the skin, of releasing them in a controlled manner. For the purposes of the present invention, it has proven particularly advantageous if the incorporated auxiliaries are osmoprotectants, such as, for example, trimethylglycine, arginine, creatine, potassium lactate, taurine, trehalose or myoinositol, which can be used in quantities from 0.01 up to 40 weight %, preferably up to 25 weight %, based on the total formulation.

The interpenetrating polymer network may be present in ratios of 1:99 to 99:1 (based on the anionic polyamino acid matrix and the polysaccharide), preferably within the ratio range of 90:10 to 10:90, and particularly preferably, within the ratio range of 80:20 to 20:80.

The mixtures obtained in this way can, depending on the compositions, be variably adjusted with regard to their effectiveness and allow adaptation to different skin types.

Osmoprotectants, so called compatible solutes, are substances that protect the skin from environmental stresses like dryness, cold, heat or salt. These substances enable survival under adverse conditions. Furthermore, osmoprotectants don't interfere with the human metabolism and are highly compatible, thus they can be applied as cosmetic active ingredient

Virtually all materials interact with moisture to a greater or lesser degree. Knowledge of moisture-induced changes in material properties is an important key parameter for decisions regarding the ability of a product to be used as a means for increasing skin moisture. The representation of the change in water content of a sample depending on the relative atmospheric humidity at constant temperature is referred to as water vapor sorption isotherm. It is the simplest and also most meaningful way of documenting interactions of a material with moisture.

From the quantity and the course of the isotherms, it is possible to derive important material properties and information with regard to their handling. The processes during sorption and desorption are mostly very complex physiochemical processes for which reason the isotherms can only be determined experimentally and cannot be predicted. Sorption isotherms are the most meaningful thermodynamic single parameter for the interactions of substances with water. Shape and course of the isotherm give information about the changes within the material.

Hysteresis is the term used to refer to the continuance of an effect after the cause has been removed. It refers to a phenomenon which is characterized by a hysteresis curve; the appearance of bistable behavior is typical of hysteresis. This means that, under identical ambient conditions, the state of a substance is dependent on the past.

Based on the water-binding capacity in the case of cosmetic active ingredients, a hysteresis means that water is released during sorption-desorption processes in a delayed manner and thus the skin moisture is durably increased.

For the purposes of the present invention, it has proven advantageous if at least one polymer component, or one osmotic protective substance, displays hysteresis behavior during sorption-desorption processes. It has proven to be particularly advantageous if both at least one polymer component, and also at least one osmotic protective substance displays hysteresis behavior during sorption-desorption processes. The present invention thus provides formulations for the topical treatment of the skin which display hysteresis behavior with regard to water binding.

Anionic Polyamino Acid Matrix:

The anionic polyamino acid matrices to be used according to the present invention are constructed from anionic amino acids and constitute copolymers of anionic amino acids and further natural or synthetic amino acids. Anionic amino acids are understood as meaning carboxylate-functional amino acids which have free carboxylic acid groups. According to the invention, preference is given to glutamic acid and aspartic acid. The molecular weight of the anionic polyamino acid matrices used according to the present invention is about 15,000 g/mol or more, and is preferably in the range between about 10,000 to 2,000,000. Particular preference is given to molecular weight ranges from about 25,000 to 1,500,000.

Anionically modified polyamino acids are known from nature and are produced, for example, by various Gram-positive bacteria. Poly-gamma-glutamic acid is produced, inter alia, by Bacillus licheniformis and Bacillus subtilis var. natto. Other heterotrophic bacteria and some cyano bacteria, such as, for example, Spirulina platensis produce the polyamide polyalpha-arginyl aspartate (cyanophycine) consisting of aspartic acid and arginine.

The biotechnological production of anionic polyamino acids is likewise described in the literature. The books by M. Bovarnick et al. from the year 1942 are the earliest examples for producing polyglutamic acid using microorganisms. In addition, the biotechnological production of polyglutamic acid is known from the patent specifications JP 43-24 472 (1969) and the German laid-open specification DE-A-198 25 507 (Bayer, 1999).

Of the abovementioned anionic polyamino acids, gamma-polyglutamic acid obtainable as fermentation product is preferably used in the present invention. Polyglutamic acid is a high-viscosity, high molecular weight polymer of glutamic acid which has free carboxyl groups which impart an overall anionic character to the polymer and cause good solubility in water. Polyglutamic acid can here also be used in the form of its alkali metal salts, ammonium salts, ethanolamine salts, and calcium salts. However, preference is given to using the free acid itself or partial neutralizates thereof which have at least still 50% free carboxyl groups.

Polyglutamic acid is described as a cosmetic moisturizing factor for topical application to the skin which both directly and also indirectly positively influences the water content in the horny layer of the skin. Firstly, polyglutamic acid has film-forming properties, secondly polyglutamic acid in skin cells can increase the production of the skin's own moisturizing factor (NMF).

The use of polyglutamic acid as moisturizer for the skin is known from JP-A-59-209 635 (Ajinomoto). It is described in this reference that it imparts moderate moisture and smoothness following application of the particular cosmetic. The long-term effect to be achieved when using customary commercially available polyglutamine types, however, is still inadequate.

Polysaccharides:

Polysaccharides constitute a heterogeneous group of polymers of varying lengths and varying compositions. Basic building blocks here are, in each case, sugar moieties which are joined together via glycosidic bonds. There are alpha- and beta-glycosidic bonds, which in turn can be between a C₁ (or C₂) atom of one sugar moiety and the C₂, C₃, C₄, C₅ or C₆ of the second. If more than two linking types are combined in one molecule, branched forms arise. A polysaccharide can consist of one type of monomer (homopolymer), although it is also possible for a plurality to be involved therein (heteropolymer). The most common sugar found in polysaccharides is glucose, the polymers formed by it are called glucans.

Scleroglucan is a polysaccharide which is secreted by certain fungi such as, for example, Sclerotium rolfsii or Sclerotium glucanicum, and serves as auxiliary in the degradation of wood. Scleroglucan consists of β-1,3-glucopyranose units in the main chain, with every third unit being β-1,6-D-glycosidically joined to an additional glucose unit. In contrast to beta-glucans from sources other than fungi, these β-1,6-branches are not extended. In the native form, scleroglucan forms the triple helix characteristic of beta-glucans. Typical molecular weights are in the range from 3 to 5×10⁶, the maximum solubility in water at a neutral to weakly acidic pH is about 1.5 to 2%. It is known from the literature that scleroglucan has water-binding and thus also thickening properties. For this reason, scleroglucan is used in cosmetics, inter alia, as thickener and gelling agent. Such applications are described, for example, in EP-A-0 979 642 (L'OREAL) and in the Japanese patent JP-A-61-267 503 (SHISEIDO).

Scleroglucan is commercially available and sold under the trade name ACTIGUM™ (Degussa). Besides the function as thickener, moisturization, antiinflammation, and a pleasant feel on the skin are highlighted as properties.

Surprisingly, low molecular weight scleroglucan exhibited a higher water-retention ability and a greater improvement in skin moisture compared with high molecular weight scleroglucan.

In a preferred embodiment of the present invention, the use of depolymerized, low molecular weight polysaccharides is therefore claimed. Particular preference here is given to the use of low molecular weight depolymerized scleroglucans.

For the purposes of the present invention, low molecular weight scleroglucans consist essentially of native scleroglucans which are cleaved by acidic, alkaline, oxidative and/or enzymatic hydrolyis. For the purpose of the present invention, preference is given to scleroglucans with an average molecular weight of 100,000 to 600,000 g/mol. Particular preference is given to scleroglucans with an average molecular weight of 200,000 to 500,000 g/mol, and particularly preferably 300,000 to 400,000 g/mol. Thus, low molecular weight scleroglucans have such a small molecular size that these polysaccharides are better able to penetrate into the skin. In addition, the small molecular size also has an advantageous effect on the production of interpenetrating networks with anionic polyamino acids since the dissolution properties and compatibility with the polyamino acid matrix improve because considerably weaker thickening occurs, which in turn permits higher use concentrations.

Interpenetrating Polymer Networks:

By combining the anionically modified polyamino acid matrix with the depolymerized polysaccharide, it is possible to obtain interpenetrating polymer networks in different ways. In the simplest case, the substances here are mixed together in a suitable ratio and mixed together in a solvent such as, for example, water.

The anionic polyamino acid matrix and the polysaccharide can remain uncrosslinked, but according to the teachings of the present invention, have to form a homogeneous, interpenetrating polymer network. However, for example in the case of polyglutamic acid (PGA) and the depolymerized scleroglucan, the helix-like structures of the starting polymers can also interwine with one another. The polysaccharide is here physically captured in the polyglutamic acid matrix, and a cross-linked interpenetrating polymer network is formed via intermolecular hydrogen bridges for example.

A further production option consists in mixing the anionically modified polyamino acid matrix with the polysaccharide, if appropriate dissolving it in water and then achieving crosslinking by means of ionizing radiation. Alternatively, it is also possible to carry out processes in which the anionically modified polyamino acid matrix is firstly converted into a hydrogel structure by crosslinking with ionizing radiation, and then the reaction with the depolymerized polysaccharide takes place.

A further method consists in carrying out chemical crosslinking between anionically modified polyamino acid matrix and depolymerized polysaccharide by treating the mixture of the two polymers with a reactive bifunctional crosslinking agent which joins the carboxylic acid groups of the anionically modified polyamino acid matrix with the hydroxy groups of the depolymerized polysaccharide. Such crosslinking agents may, for example, be diepoxides, such as ethylene glycol diglycidyl ether.

Methods for determining the degree of intertwining of an interpenetrating polymer network are sufficiently known and are based on the altered mechanical and electrical properties compared with a pure mixture of the starting polymers. The swelling capacity, which correlates to the number of crosslinking units within the polymer network, can be determined using customary methods. In this regard, reference may be made to the discussion in the specialist literature under “Moleculer Interpenetrations of Sorption in Polymers Part 1”, Advances in Polymer Science Vol. 99, Springer-Verlag, Berlin, Heidelberg, Germany (pp. 22 to 36, 1991). With the addition of water, such interpenetrating polymer networks according to the invention form hydrogels which are characterized by excellent skin compatibility and ability to adhere to the skin without being sticky. These interpenetrating networks can be charged with low molecular weight substances such as, for example, osmoprotectants.

### APPLICABILITY

Cosmetic or dermatological preparations according to the present invention for regulating skin moisture are understood primarily as meaning those compositions which are applied to the facial skin and/or other parts of the body.

These compositions are the customarily used cosmetic or dermatological formulations, which are generally in the form of aqueous alcoholic solutions, creams, emulsions, lotions, gels, aerosol spray or aerosol foam, nonaerosol spray or nonaerosol foam. Accordingly, to be adapted, the compositions can also include further customary constituents which serve for the treatment, care, cleansing and protection of the skin, such as, for example, skin cosmetic active ingredients such as, for example, ceramides, pseudoceramides, protein hydrolysates of vegetable or animal origin based on keratin, collagen, elastin, wheat, rice, soya, milk silk corn, amino acids and amino acid derivatives, anti-inflammatory active ingredients, antimicrobial active ingredients, customary antioxidants, vitamins, dexpanthenol, lactic acid, pyrrolidonecarboxylic acid, bisabolol, and plant, yeast and algae extracts.

The combination with customarily used organic or inorganic UV filter substances is likewise possible and to be regarded, for example, in sunscreen preparations, as particularly advantageous since during use, drying-out of the skin can be effectively suppressed and as a result the natural protective function of the skin is retained. In addition, further cosmetic auxiliaries and additives which are customary in such preparations may also be present Such auxiliaries are, for example, solubilizers, such as ethanol, isopropanol, ethylene glycol propylene glycol, glycerol and diethylene glycol. Further components include cosmetic oils of vegetable and synthetic origin, emollients, fats, waxes, refatting agents, emulsifiers, thickeners, anionic, zwitterionic, ampholytic and nonionic surfactants, and also fragrances and preservatives.

Finally, the formulations according to the present invention can also comprise complexing agents, such as EDTA, NTA, β-alaninediacetic acid and phosphonic acid, dyes for coloring the cosmetic preparation, opacifiers such as latex, styrene/PVP and styrene/acrylamide copolymers, pearlising agents such as ethylene glycol mono- and distearate and PEG-3 distearate, pigments, photoprotective agents, thickeners or propellants.

Typical guide formulations for skin-treatment compositions belong to the known prior art and are contained, for example, in brochures from the manufacturer of the respective basic and active ingredients. One informative source of formulations of this type is, for example, the Kosmetik-Jahrbuch published annually (editor B. Ziolkowsky, Verlag für Chemische Industrie).

These existing formulations can generally be adopted unchanged. If required, for adaptation and optimization, the desired modifications can, however, be undertaken without complications by simple experiments.

The formulations according to the present invention are prepared in the usual way, with the moisturizer mixture preferably being dissolved in the aqueous phase of the formulation, or being added at temperatures below 40° C. after the formulation has been prepared. The pH is preferably adjusted last by adding the acid and/or the buffer mixture intended for this.

The following examples are provided to illustrate the active ingredient combination of the present application and to demonstrate its use in cosmetic and/or dermatological formulations.

### EXAMPLE 1

Preparation of an Active Ingredient Combination of the Present Invention

The inventive active ingredient combination was prepared as follows: water was initially introduced and all of the water-soluble constituents, such as the osmoprotectants, with the exception of the gamma-polyglutamic acid and of the low molecular weight depolymerized scleroglucan, were dissolved. The solution was then heated to about 60°-70° C., and the polyglutamic acid and the scleroglucan were added with considerable shearing (Ystrahl mixer, 10-20000 min⁻¹). Stirring was carried out with the highest shearing possible until the solution was homogeneous and then it was cooled with gentle stirring to 300-40° C.

Active ingredient combination 1

| gamma-Polyglutamic acid | |
|---|---|
| Anionic polyamino acid matrix, component a) | 2.0% |
| Depolymerized low molecular weight scleroglucan (average | 2.0% |
| molecular weight about 350,000) (polysaccharide, component b) | |
| Trimethylglycine (component c) | 20.0% |
| Urea | 10.0% |
| Potassium lactate (component c) | 5.0% |
| Water | 61.0% |

### EXAMPLE 2

Preparation of a Skincare Formulation

The skincare formulation can be formulated as an aqueous lotion, a water-in-oil or oil-in-water emulsion, an oil or oil-alcohol lotion, a vesicular dispersion of anionic or nonionic amphiphilic lipids, an aqueous, water-alcohol, an alcohol or oil-alcohol gel, a solid stick or as an aerosol.

If it is formulated as water-in-oil or oil-in-water emulsion, the cosmetically acceptable carrier preferably comprises 5 to 50% of an oil phase and 47 to 94.95% water, based on the total amount of the formulation.

The oil phase can comprise any cosmetic oil or a mixture thereof. Examples of such oils include aliphatic hydrocarbons, such as liquid paraffin, squalane, Vaseline and ceresin, vegetable oils, such as olive oil, almond oil, sesame oil, avocado oil, castor oil, cocoa butter and palm oil, animal oils, such as shark liver oil, cod liver oil, whale oil, beef tallow and butterfat, waxes such as beeswax, carnauba palm wax, spermaceti and lanolin, fatty acids, such as lauric acid, myristic acid, palmitic acid, stearic acid, oleic acid and behenic acid; aliphatic alcohols, such as lauryl, stearyl, cetyl and oleyl alcohol and aliphatic esters, such as isopropyl, isocetyl or octadecyl myristate, butyl stearate, hexyl laurate, diisopropyl ester of adipic acid or diisopropyl sebacate. Preferred mono- or polyols for use in oil-alcohol lotions or oil-alcohol gel or alcohol gel include ethanol, isopropanol, propylene glycol, hexylene glycol, glycerol and sorbitol.

O/W cream with active ingredient combination 1

| | | | |
|---|---|---|---|
| A) | TEGO ® Care PS | Methyl glucose | 3.0% |
| | | sesquistearate | |
| | TEGO ® Alkanol 18 | Stearyl alcohol | 1.5% |
| | TEGIN ® M | Glyceryl stearate | 3.5% |
| | TEGOSOFT ® liquid | Cetearyl | 9.2% |
| | | ethylhexanoate | |
| | TEGOSOFT ® CT | Caprylic/Capric | 10.0% |
| | | triglyceride | |
| B) | Glycerol | | 3.0% |
| | Water | | 62.65% |
| C) | TEGO ® Carbomer 134 | Carbomer | 0.2% |
| | TEGOSOFT ® liquid | Cetearyl | 0.8% |
| | | ethylhexanoate | |
| | Active ingredient combination 1 | | 5.0% |
| | NaOH (10%) | | 0.65% |
| | Perfume | | 0.5% |
| | Preservative | | q.s. |

Preparation of the Formulation:

Phase A) and B) were heated to 65° C. Phase B) was then added to A) and homogenized (e.g., using an Ultra Turrax). The emulsion was cooled to 30° C. with gentle stirring. At 60° C. phase C) was added and the mixture was briefly homogenized again. The active ingredient combination 1 and also the sodium hydroxide solution and the perfume oil were added one after the other at 35°-40° C.

For the efficacy studies described below, an O/W cream of simple formulation which had the following composition was used:

TABLE 1

| | |
|---|---|
| Ceteareth-25 | 2.0% |
| Glyceryl stearate | 4.0% |
| Stearyl alcohol | 2.0% |
| Ethylhexyl stearate | 8.5% |
| Caprylic/capric triglyceride | 8.5% |
| Preservative | 0.1% |
| Water | 64.9% |
| Active ingredient combination 1 | 10.0% |

### EXAMPLE 3

Determination of the Moisturizer Properties of the Active Ingredient Combination

Corneometry is a noninvasive method which permits conclusions relating to the skin moisture on the basis of changes in the physical parameters within the stratum corneum. In practice, 12 to 15 subjects are recruited whose skin moisture is measured before and after single application of the test formulations using a HM99 corneometer (Courage & Khazaka, Cologne, Germany) according to the manufacturer's instructions.

After climatization of the subjects over 15 minutes under defined conditions of 22° C. and 55% relative humidity, the skin hydration is determined corneometrically and afterwards 50 mg of the test formulation are applied to a skin area each with a diameter of 2.5 cm, and after 2 minutes any residues of the formulation are removed.

After a contact time of two hours, the skin hydration is measured again following climatization of 15 minutes (22° C., 55% rel. humidity). An increase of the relative corneometer units CU correlates directly with improved skin hydration.

The concentrations of the individual substances were chosen so that they correspond to the concentration in 10% active ingredient combination 1. The table below (Table 2) shows the increase in the relative corneometer units (CU) compared with the blank formulation.

TABLE 2

| | ΔCU |
|---|---|
| 0.2% scleroglucan | 1.1 |
| 0.2% gamma-polyglutamic acid | 1.6 |
| 2.0% trimethylglycine | 3.2 |
| 1.0% urea | 3.6 |
| 0.5% potassium lactate | 1.6 |
| Active ingredient combination 1 consisting | 14.2 |
| of: | |
| 0.2% scleroglucan | |
| 0.2% gamma-polyglutamic acid | |
| 2.0% trimethylglycine | |
| 1.0% urea | |
| 0.5% potassium lactate | |

It is seen that even the individual substances bring about an improvement in the moisture state of the skin. By combining the different ingredients, this hydration was significantly and synergistically increased. Whereas the sum of the individual components suggested a theoretical improvement in the skin moisture by 11.1 corneometer units, the improvement in reality is 14.2 corneometer units. This corresponds to a synergistic improvement as a result of the combination of the active ingredients of 27.9%.

### EXAMPLE 4

Determination of the In Vitro Sorption-Desorption Properties

In order to determine the in vitro sorption-desorption properties of moisturizers, these were firstly dried overnight at 80° C. and 0% relative humidity (r.h.) and then cooled to room temperature in a desiccator. 4-5 g of the moisturizer substance were then placed into a Petri dish and stored for 24 h at 20% rh. The weight increase was recorded and the atmospheric humidity was then increased by 10%. After 24 h, the weight of the moisturizer was monitored again. This was repeated in stages to an atmospheric humidity of 80%. The atmospheric humidity was then reduced again every 24 h in 10% stages and the weight decrease was recorded.

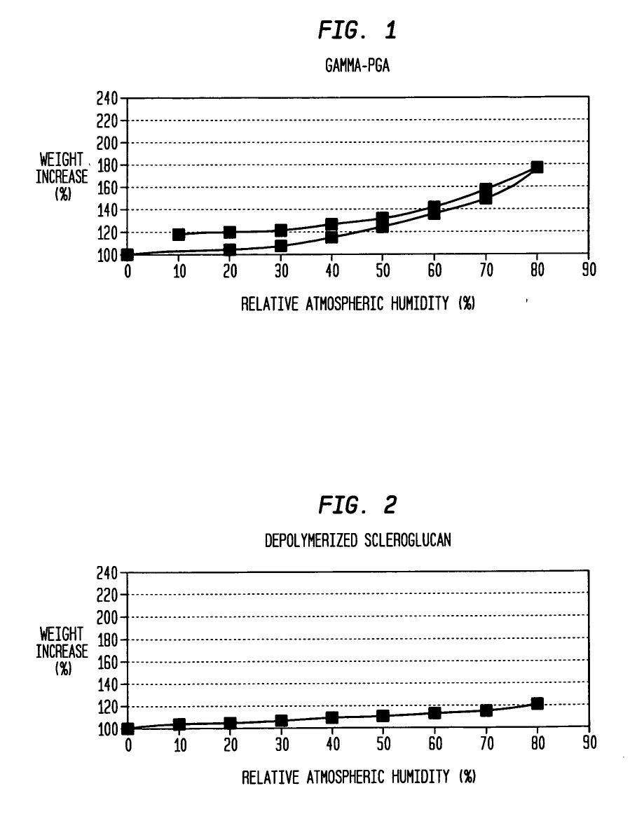

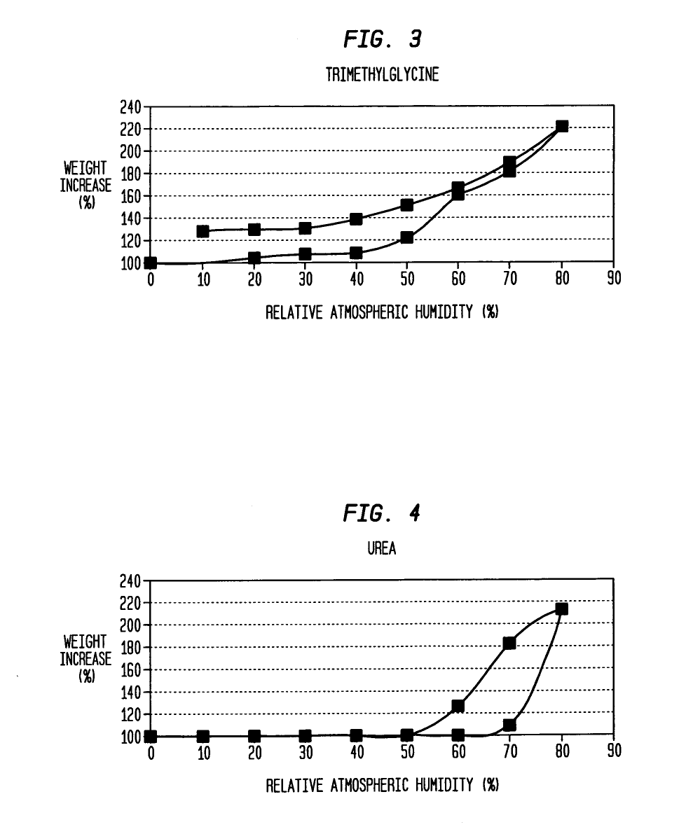

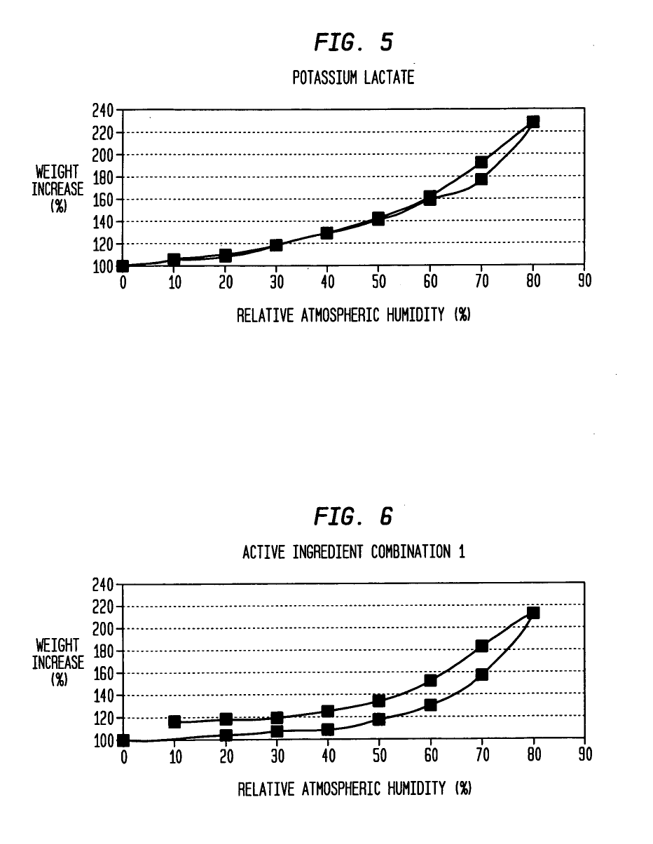

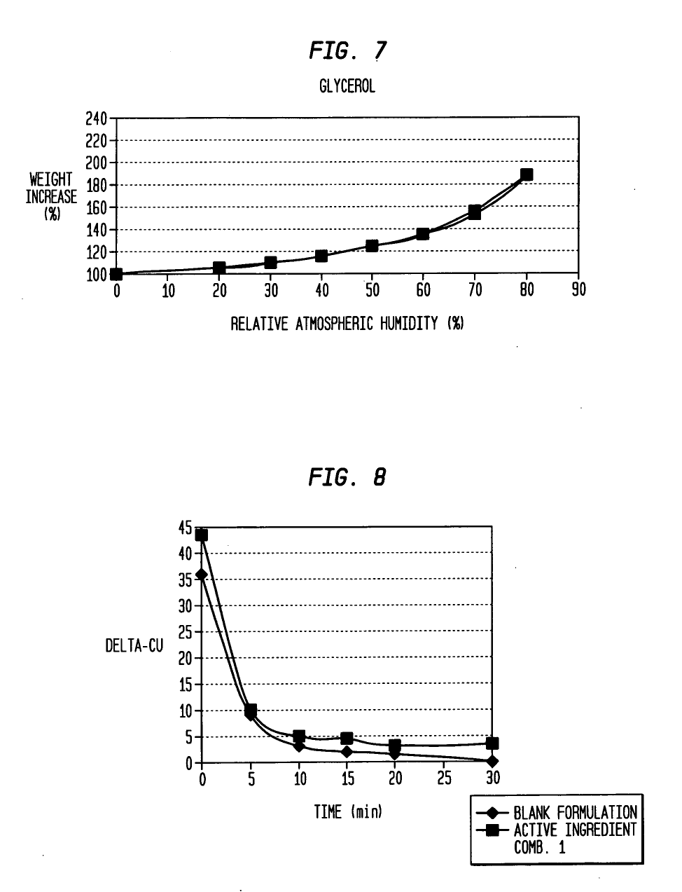

A good moisturizer is expected to absorb the largest possible amounts of water as the atmospheric humidity increases. This was the case, for example, with glycerol. It should, however, also be able to store the absorbed water as the atmospheric humidity decreases, which glycerol is not able to do. Considering, for example, the sorption-desorption curve of polyglutamic acid, of trimethylglycine and also of urea, it was seen that, besides a very good ability to absorb water, these substances were also able to hold onto the absorbed water as the atmospheric humidity decrees. This hysteresis behavior was advantageous for maintaining skin moisture through topical application and for an active ingredient should extend over the largest possible moisture range. By combining the ingredients, this was the case for active ingredient combination 1. FIGS. 1-7 are graphs showing the results of this particular study. In each of FIGS. 1-7, the active ingredient is mentioned at the top of each graph, the y-axis represents the weight increase, %, and the x-axis represents the relative atmospheric humidity, %.

### EXAMPLE 5

In Vivo Sorption-Desorption

To determine the in vivo sorption-desorption properties of the active ingredient combination 1, the O/W cream containing 10% active ingredient combination 1 (for composition see Table 1) was applied twice daily by 3 subjects to the forearm for 3 weeks. After 3 weeks, the hydration state of the skin was firstly determined by means of corneometer HM99. A wet paper towel was then placed onto the forearm (contact time: 2 min) in order to saturate the upper layers of the skin with moisture. The skin surface was then patted with a dry paper towel and the skin moisture was measured. The skin moisture was then monitored at regular intervals.

As FIG. 8 shows, the skin which had been treated with the active ingredient combination 1 firstly absorbed more water and secondly also binded this water more strongly and for a longer period of time. This result shows the in vivo effect of the results from example 4.

### EXAMPLE 6

Long-Term Test

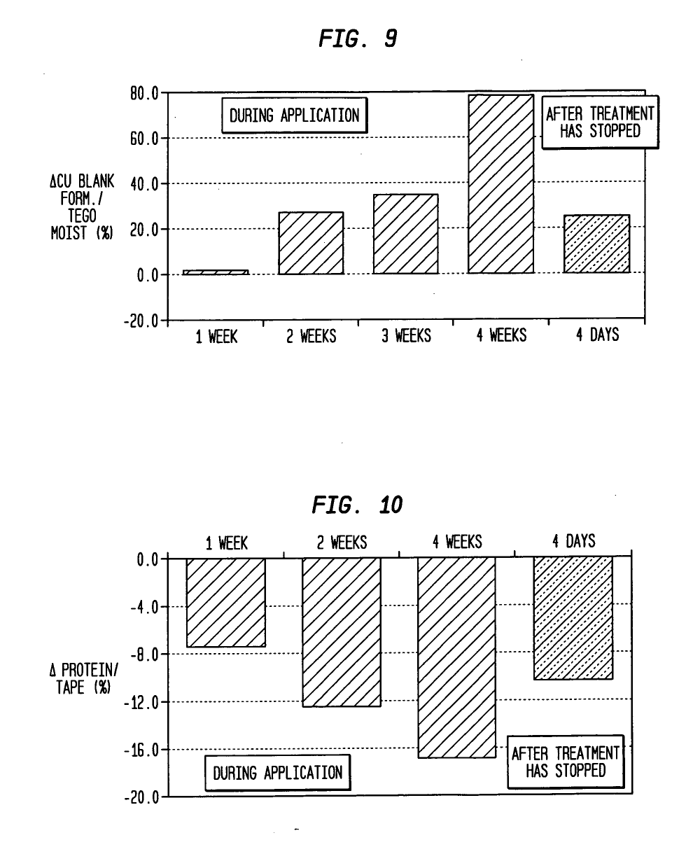

The O/W cream (see Table 1) containing 10% active ingredient combination, and the corresponding blank formulation were applied twice daily by 14 subjects to the forearm over a period of 4 weeks. At the start of the test, and at one-week intervals during the treatment phase, the skin moisture was monitored using the HM99 corneometer. After 4 weeks, the treatment was stopped and the skin moisture after 4 days was monitored. FIG. 9 shows the increase in the relative corneometer units by the active ingredient combination 1 compared to the blank formulation.

As the treatment time increases, hydration of the skin by the active ingredient combination 1 improved significantly compared to the blank formulation This improvement can also still be detected 4 days after treatment had stopped.

During the 4-week application, the roughness of the skin and the appearance of the skin were also assessed. This was carried out using D-squame tapes (transparent sticky tapes), which were stuck onto the forearm with a defined pressure and then removed again in order, in so doing, to remove the uppermost loose flakes of skin. The amount of skin flakes was determined quantitatively by treating the D-squame tapes with NaOH in order to dissolve the proteins. HCl was then used for neutralization and the concentration of the dissolved proteins was determined by means of the Bradford test. The results of this test is shown, for example, in FIG. 10.

Specifically, FIG. 10 shows the difference in the protein concentration between the skin treated with active ingredient combination 1 and the skin treated with the blank formulation. The lower the protein concentration, the less rough the surface of the skin.

As the treatment time increases, the roughness of the skin becomes significantly less through treatment with the active ingredient combination 1 compared with the blank formulation. The roughness of the skin changes only slightly even after the treatment has stopped, i.e., the care effect of active ingredient combination 1 persists.

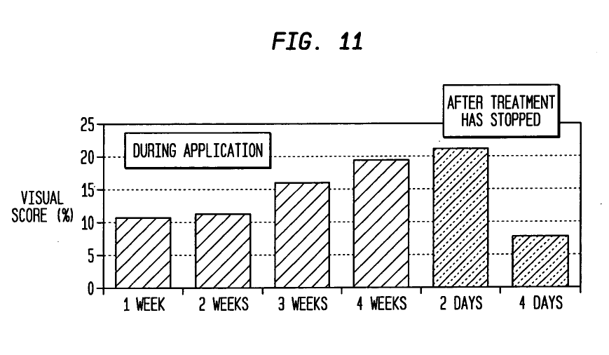

This was also confirmed by the visual assessment of the size of the skin flakes on the D-squame tapes. For this, the tapes were visually assessed by reference to a scale from 1-5 (1=very fine flakes, 5=very large flakes). FIG. 11 shows the improvement in the skin treated with active ingredient combination 1 compared with skin which was treated with the blank formulation.

The structure of the skin improved significantly during the treatment period. This effect was retained even after the treatment has stopped.

### EXAMPLE 7

Influence on the Skin Elasticity

To determine the positive effect of active ingredient combination 1 on the skin elasticity, the O/W cream described in Table 3 containing 5% active ingredient combination was applied twice daily by 12 subjects to the inside of the forearm for 4 weeks. At the start of the test and also after 4 weeks, the skin elasticity was monitored using a cutometer MPA 580 from Courage & Khazaka.

When determining the skin elasticity using a cutometer, the skin was sucked under subatmospheric pressure (450 mbar) into a probe aperture. The diameter of this probe aperture was 2 mm. The extension of the skin was maintained for 5 s, then the skin was released again by raising the subatmospheric pressure. This cycle of extension and release was repeated three times. The curve obtained can be used, inter alia, to calculate the following elasticity parameters:
the parameter R1 was the height of the residual deformation after the first measurement cycle. It thus described the ability of the skin to recover.
R2 was the so-called gross elasticity, the quotient of UA and UF, where UF was the maximum distention of the skin at the end of the first measurement cycle, and UA was the difference in UF and R1.
R5 was the so-called net elasticity, the quotient of UR and UE, where UR was the height of the elastic extension upon release, UE was the height of the elastic extension of the skin when sucked into the probe.
R7 was the quotient of UR and UF, i.e., it represented the ratio of the elastic part after switching off the vacuum to the total deformation of the first cycle.
F0 was the area within the rectangle formed from UF×sucking time, above the curve. A completely elastic material would have no area.

Further information on determining the skin elasticity using a cutometer can be found in the operating instructions for the cutometer (information and directions for use for Cutometer MPA 580, CK electronic GmbH, Cologne).

TABLE 3
O/W cream

| | |
|---|---|
| Ceteareth-25 | 2.0% |
| bis-PEG/PPG-16/16 PEG/PPG dimethicone; | 1.0% |
| Caprylic/capric triglyceride | |
| Stearic acid | 0.5% |
| Cetearyl alcohol | 5.0% |
| C12-15 alkyl benzoate | 9.0% |
| PPG-3 myristyl ether | 9.0% |
| Water | 67.32% |
| NaOH (10%) | 0.43% |
| Carbomer | 0.15% |
| Ethylhexyl palmitate | 0.60% |
| Active ingredient combination 1 | 5.0% |

The table below shows the improvement in the various elasticity parameters after applying the test formulation for 4 weeks.

TABLE 4
Improvement in the elasticity parameters after applying the test
formulations for four weeks.

| Elasticity parameter | Excipient | 5% active ingredient combination 1 |
|---|---|---|
| R1 | 0.0% | 21.8% |
| R2 | −0.4% | 1.6% |
| R5 | −1.5% | 2.3% |
| R7 | −2.4% | 0.7% |
| F0 | 4.0% | 9.9% |

In the case of parameters R1 and F0, there was a significant improvement in the skin elasticity as a result of active ingredient combination 1. Considering parameters R2, R5 and R7, the excipient brought about a slight deterioration, while the active ingredient combination had a slightly positive effect on the skin elasticity.

While the present invention has been particularly shown and described with respect to preferred embodiments thereof, it will be understood by those skilled in the art that the foregoing and other changes in forms and details may be made without departing from the spirit and scope of the present invention. It is therefore intended that the present invention not be limited to the exact forms and details described and illustrated, but fall within the scope of the appended claims.

What is claimed is:

1. A cosmetic and/or dermatological formulation for local topical treatment of parts of a body for regulating and improving moisture content of skin, comprising as an active ingredient combination for topical application:
a) at least one anionic polyamino acid matrix, and
b) at least one polysaccharide.

2. The cosmetic and/or dermatological formulation according to claim 1, further comprising c) one or more osmoprotectants.

3. The cosmetic and/or dermatological formulation according to claim 1, wherein the active ingredient combination of a) and b) is present in the form of an interpenetrating polymer network.

4. The cosmetic and/or dermatological formulation according to claim 1, consisting of an active ingredient combination of at least one polysaccharide matrix and at least one anionic polyamino acid matrix which has hysteresis behavior with regard to water binding.

5. The cosmetic and/or dermatological formulation according to claim 1, wherein the anionic polyamino acid is a polyglutamic acid with an average molecular weight of from 1,000,000 to 2,000,000 g/mol.

6. The cosmetic and/or dermatological formulation according to claim 1, wherein the polysaccharide is scleroglucan, hyaluronic acid, beta-glucan, cellulose, derivatized cellulose, amylose, amylopectin or xanthan.

7. The cosmetic and/or dermatological formulation according to claim 1, wherein the polysaccharide is scleroglucan with an average molecular weight of less than 600,000 g/mol.

8. The cosmetic and/or dermatological formulation according to claim 7, wherein said average molecular weight is less than 500,000 g/mol.

9. The cosmetic and/or dermatological formulation according to claim 8, wherein said average molecular weight is less than 400,000 g/mol.

10. The cosmetic and/or dermatological formulation according to claim 1, wherein components a) and b) are used in a ratio from 1/99 to 99/1.

11. The cosmetic and/or dermatological formulation according to claim 2, wherein the osmoprotectant c) used is at least one compound selected from the group consisting of trimethylglycine, arginine, creatine, potassium lactate, taurine, trehalose, and myoinositol.

12. The cosmetic and/or dermatological formulation according to claim 1 further comprising at least one compound selected from the group consisting of vitamins, peptides, ceramides, sphingolipids, antioxidants and mixtures thereof.

13. The cosmetic and/or dermatological formulation according to claim 1 further comprises one further component selected from the group consisting of urea, glycerol, niacinamide, panthenol, allantoin, bisabolol, pyrrolidonecarboxylic acid, proteins, protein hydrolysates, amino acids, propylene glycol, phospholipids, lysophospholipids, plant extracts, bacterial extracts and fungal extracts.

14. A cosmetic composition comprising
a) a polyglutamic acid in an amount between 0.05 and 5%,
b) at least one depolymerized scleroglucan in an amount between 0.05 and 5%, and
c) at least one osmoprotectant in an amount between 0.01 and 25%.
